SEQUENCE LISTING (73 sequences)

SEQ ID NO: 1 - DNA, 1345 bp, Homo sapiens
ctgcagtgaccactgccccatcattgctggctgaggtggttggggtccatctggctatctgggcagctgttctcttctctcctttctctcctgtttccagacatgcagtatttccagagagaaggggccactctttggcaaagaacctgtctaacttgctatctatggcaggacctttgaagggttcacaggaagcagcacaaattgatactattccaccaagccatcagctccatctcatccatgccctgtctctcctttaggggtccccttgccaacagaatcacagaggaccagcctgaaagtgcagagacagcagctgaggcacagccaagagctctggctgtattaatgacctaagaagtcaccagaaagtcagaaggatgcatagcagaggcccagcaatctcagctaagtcaactccaccagcctttctagttgcccactgtgtgtacagcacsctggtagggaccagagccatgacagggaataagactagactatgcccttgaggagctcacctctgttcagggaaacaggcgtggaaacacaatggtggtaaagaggaaagaggacaataggattgcatgaaggggatggaaagtgcccaggggaggaaatggttacatctgtgtgaggagtttggtgaggaaagactctaagagaaggctctgtctgtctgggtttggaaggatgtgtaggagtcttctagggggcacaggcacactccaggcataggtaaagatctgtaggtgtggcttgttgggatgaatttcaagtattttggaatgaggacagccatagagacaagggcargagagaggcgatttaatagattttatgccaatggctccacttgagtttctgataagaacccagaacccttggactccccagtaacattgattgagttgtttatgatacctcatagaatatgaactcaaaggaggtcagtgagtggtgtgtgtgtgattctttgccaacttccaaggtggagaagcctcttccaactgcaggcagagcacaggtggccctgctactggctgcagctccagccctgcctccttctctagcatataaacaatccaacagcctcactgaatcactgctgtgcagggcaggaaagctccatgcacatagcccagcaaagagcaacacagagctgaaaggaagactcagaggagagagataagtaaggaaagtagtgatggctctcatcccagacttggccatggaaacctggcttctcctggctgtcagcctggtgctcctctatctgtgagtaactgttcaggctcctcttctctgtttcttggacttggggtcgtaatcaggcctctctttt

SEQ ID NO: 2 - DNA, 19 bp, Artificial Sequence
  Oligonucleotide of CYP3A4 region
acaagggcaagagagaggc

SEQ ID NO: 3 - DNA, 19 bp, Artificial Sequence
  Oligonucleotide of CYP3A4 region
acaagggcaggagagaggc

SEQ ID NO: 4 - DNA, 10 bp, Artificial Sequence
  Oligonucelotide of CYP3A4 region
agggcaagag

SEQ ID NO: 5 - DNA, 10 bp, Artificial Sequence
  Oligonucleotide of CYP3A4 region
agggcaggag

SEQ ID NO: 6 - DNA, 2438 bp, Homo sapiens
gatctcagatatcccttctatctacacattatctataattctttctttctgtaaactgaaaggtcctagaaggagccgcagctcagcaggagagaggaggagctgagctgggacccctacctcctgaggaatgaaatgattattataaagacagcaaccgagcttattttacccaaaataaggtagtatatttctgttagagtttagagtttcatgagtcagggaccaagttattgcttttctttgccctgtataaaggcttctccaaggcctttgacttacctaagtactaaatgttataaaaccaaactcttctgacctctcaatctagtcaactggggctgtaattattaatgaaattaatgtttattttgaaaataatttactagactgaattacgaaatcctgaatcattgtacactatcagtaaatattggtggacccaactgaactgaatgttttgcttgaaatgaaacctttgagatgcagggcttatgggttctagtcccagctctagcactagcagacagcatgttcttggctaagatactgaatcttcaaggctcagcttcctcattccggaaatgggtcaattttattgtaagcagaggtaattgagagattcaaaagggacatgaggtgtaacaattctctgtaaattgttagaatccctgttaaaaatgaccagtaaagctttgtgcaactgtgtcttgacataactttatttttcttaataaaagaaatggaaataacctcactagggaatttagaacaaatatgatgatatctttaaagaaaatggctttgcacaagtattgacattaatgatctagtaaagtgtatctttctagttgtatttagatcctcaactcagtatgtcagctcctgttaaggtctatacattgtggtggttctgtgctgtgggtccatttagtgatttccctacctcccatcttytattgcatccacaactgtggttctgtccataatttcctttgctttctgtgcattattacatcatatctgaaaatgagaaaccaaaaacaatrgaaagcagccatgtctggaggtgactggggggtcgagaagccctagtttctcaaacccttagcaccaaatttttccctcagttacactgagcgtttcacttctgcagtgatggaraagggagatcccttatttcttctcatgagcatctctggtgctgtttcccttagagacaaataaggggttctatttaatgtgaagcctgttttatgaacagaataaatgtggtgtatattcagaataactaatgtttggaagttgttttatttttgctaaaaattgttctcaaggcagctctggtgtaagagataatacaccacgatgggcatcagaagacctcagctcaaatcccagttctgccagctatgagctgtgtggcaccaacaggtgtcctgttctcccagggtctcccttttcccatttgaaaaataaaaaataacaattcctgccttcaggaattttttttagggggtttaatkgtaaaggtgtttatatctgctaaggtaatttacttgatatatgtttggttatttaagatatatgagttatgttagctatttcatgtttaggctgctgtatttttagtaggctatattaaatatttgaaaggattwmattataaagaacaaagtctcctaatctttgatatagcattgacatactttttaaatatacaaggcatagaatatggccatttctgttaaatcatatattcccaactggttattaatctaagaattcagaattttgagtaattgcttttgcatcagattatttacttcagtgctctcaattatgatggtgcattagaaccatctgggttaacatttgttttttattaccaatacctaggctccaaccaagtacagtgaaactggaatgtacagagtggacaatggaacgaaggagaacaagaccaaaggacattttatttttatctgtatcagtgggtcaaagtcctttcagaaggagcatatagtggacctaggtgattggtcaatttatccatcaaagaggcacacaccgaattagcatggagtgttataaaaggcttggagtgcaagctcatggttgtcttaacaagaagagaaggcttcaatggattctcttgtggtccttgtgctctgtctctcatgtttgcttctcctttcactctggagacagagctctgggagaggaaaactccctcctggccccactcctctcccagtgattggaaatatcctacagataggtattaaggacatcagcaaatccttaaccaatgtaagtatgctccttcagtggcttgcaaaaggtaagtaaattcacctgtattttttaaataaagtgtatccctagaggtacatgttacaagaggtaatggtaaagtaaaatactttgaaaggctt

SEQ ID NO: 7 - DNA, 20 bp, Artificial Sequence
  Primer
ccagcctgaaagtgcagaga

SEQ ID NO: 8 - DNA, 25 bp, Artificial Sequence
  Primer
tcttagagtctttcctcaccaaact

SEQ ID NO: 9 - DNA, 20 bp, Artificial Sequence
  Primer
catgccctgtctctccttta

SEQ ID NO: 10 - DNA, 19 bp, Artificial Sequence
  Primer
ccatccccttcatgcaatc

SEQ ID NO: 11 - DNA, 11 bp, Artificial Sequence
  Oligonucleotide representing the coding strand complementary to the polymorphic site 461
agcaccctggt

SEQ ID NO: 12 - DNA, 11 bp, Artificial Sequence
  Oligonucleotide representing the coding strand complementary to the polymorphic site 461
agcacgctggt

SEQ ID NO: 13 - DNA, 11 bp, Artificial Sequence
  Oligonucleotide representing the non-coding strand complementary to the polymorphic site 461
accagggtgct

SEQ ID NO: 14 - DNA, 11 bp, Artificial Sequence
  Oligonucleotide representing the non-coding strand complementary to the polymorphic site 461
accagcgtgct

SEQ ID NO: 15 - DNA, 11 bp, Artificial Sequence
  Oligonucleotide of the novel polymorphic site 461 on the coding strand
gtgtgtacagc

SEQ ID NO: 16 - DNA, 11 bp, Artificial Sequence
  Oligonucleotide of the novel polymorphic site 461 on the non-coding strand
gctgtacacac

SEQ ID NO: 17 - DNA, 11 bp, Artificial Sequence
  Oligonucleotide of the novel polymorphic site 461 on the non-coding strand
tggtccctacc

SEQ ID NO: 18 - DNA, 11 bp, Artificial Sequence
  Oligonucleotide of the novel polymorphic site 461 on the coding strand
ggtagggacca

SEQ ID NO: 19 - DNA, 25 bp, Artificial Sequence
  Primer
cactagggaatttagaacaaatatg

SEQ ID NO: 20 - DNA, 23 bp, Artificial Sequence
  Primer
gcacagaaagcaaaggaaattat

SEQ ID NO: 21 - DNA, 27 bp, Artificial Sequence
  Primer
tgtatttagatcctcaactcagtatgt

SEQ ID NO: 22 - DNA, 21 bp, Artificial Sequence
  Primer
ggatctcccttctccatcact

SEQ ID NO: 23 - DNA, 23 bp, Artificial Sequence
  Primer
ggtccatttagtgatttccctac

SEQ ID NO: 24 - DNA, 25 bp, Artificial Sequence
  Primer
atacaccacatttattctgttcata

SEQ ID NO: 25 - DNA, 22 bp, Artificial Sequence
  Primer
ccaaatttttccctcagttaca

SEQ ID NO: 26 - DNA, 20 bp, Artificial Sequence
  Primer
ttggtgccacacagctcata

SEQ ID NO: 27 - DNA, 20 bp, Artificial Sequence
  Primer
gccttcaggaatttttttta

SEQ ID NO: 28 - DNA, 25 bp, Artificial Sequence
  Primer
ccagttgggaatatatgatttaaca

SEQ ID NO: 29 - DNA, 25 bp, Artificial Sequence
  Primer
gctgctgtatttttagtaggctata

SEQ ID NO: 30 - DNA, 22 bp, Artificial Sequence
  Primer
cgttccattgtccactctgtac

SEQ ID NO: 31 - DNA, 20 bp, Artificial Sequence
  Primer
tcaaggcagctctggtgtaa

SEQ ID NO: 32 - DNA, 25 bp, Artificial Sequence
  Primer
agttgggaatatatgatttaacaga

SEQ ID NO: 33 - DNA, 11 bp, Artificial Sequence
  Oligonucleotide representing the coding strand
atcttctattg

SEQ ID NO: 34 - DNA, 11 bp, Artificial Sequence
  Oligonucleotide representing the coding strand
atcttttattg

SEQ ID NO: 35 - DNA, 11 bp, Artificial Sequence
  Oligonucleotide representing the coding strand
acaatagaaag

SEQ ID NO: 36 - DNA, 11 bp, Artificial Sequence
  Oligonucleotide representing the coding strand
acaatggaaag

SEQ ID NO: 37 - DNA, 11 bp, Artificial Sequence
  Oligonucleotide representing the coding strand
atggagaaggg

SEQ ID NO: 38 - DNA, 11 bp, Artificial Sequence
  Oligonucleotide representing the coding strand
atggaaaaggg

SEQ ID NO: 39 - DNA, 11 bp, Artificial Sequence
  Oligonucleotide representing the coding strand
ttaatggtaaa

SEQ ID NO: 40 - DNA, 11 bp, Artificial Sequence
  Oligonucleotide representing the coding strand
ttaattgtaaa

SEQ ID NO: 41 - DNA, 12 bp, Artificial Sequence
  Oligonucleotide representing the coding strand
ggatttcattat

SEQ ID NO: 42 - DNA, 12 bp, Artificial Sequence
  Oligonucleotide representing the coding strand
ggattaaattat

SEQ ID NO: 43 - DNA, 11 bp, Artificial Sequence
  Oligonucleotide representing the non-coding strand
caatagaagat

SEQ ID NO: 44 - DNA, 11 bp, Artificial Sequence
  Oligonucleotide representing the non-coding strand
caataaaagat

SEQ ID NO: 45 - DNA, 11 bp, Artificial Sequence
  Oligonucleotide representing the non-coding strand
ctttctattgt

SEQ ID NO: 46 - DNA, 11 bp, Artificial Sequence
  Oligonucleotide representing the non-coding strand
ctttccattgt

SEQ ID NO: 47 - DNA, 11 bp, Artificial Sequence
  Oligonucleotide representing the non-coding strand
cccttctccat

SEQ ID NO: 48 - DNA, 11 bp, Artificial Sequence
  Oligonucleotide representing the non-coding strand
cccttttccat

SEQ ID NO: 49 - DNA, 11 bp, Artificial Sequence
  Oligonucleotide representing the non-coding strand
tttaccattaa

SEQ ID NO: 50 - DNA, 11 bp, Artificial Sequence
  Oligonucleotide representing the non-coding strand
tttacaattaa

SEQ ID NO: 51 - DNA, 12 bp, Artificial Sequence
  Oligonucleotide representing the non-coding strand
ataatgaaatcc

SEQ ID NO: 52 - DNA, 12 bp, Artificial Sequence
  Oligonucleotide representing the non-coding strand
ataatttaatcc

SEQ ID NO: 53 - DNA, 11 bp, Artificial Sequence
  5′-sequence to the polymorphic sites on the coding strand
tacctcccatc

SEQ ID NO: 54 - DNA, 11 bp, Artificial Sequence
  5′-sequence to the polymorphic sites on the coding strand
aaccaaaaaca

SEQ ID NO: 55 - DNA, 11 bp, Artificial Sequence
  5′-sequence to the polymorphic sites on the coding strand
ctgcagtgatg

SEQ ID NO: 56 - DNA, 11 bp, Artificial Sequence
  5′-sequence to the polymorphic sites on the coding strand
tagggggttta

SEQ ID NO: 57 - DNA, 11 bp, Artificial Sequence
  5′-sequence to the polymorphic sites on the coding strand
atttgaaagga

SEQ ID NO: 58 - DNA, 11 bp, Artificial Sequence
  5′-sequence to the polymorphic sites on the non-coding strand
gatgggaggta

SEQ ID NO: 59 - DNA, 11 bp, Artificial Sequence
  5′-sequence to the polymorphic sites on the non-coding strand
tgtttttggtt

SEQ ID NO: 60 - DNA, 11 bp, Artificial Sequence
  5′-sequence to the polymorphic sites on the non-coding strand
catcactgcag

SEQ ID NO: 61 - DNA, 11 bp, Artificial Sequence
  5′-sequence to the polymorphic sites on the non-coding strand
taaacccccta

SEQ ID NO: 62 - DNA, 11 bp, Artificial Sequence
  5′-sequence to the polymorphic sites on the non-coding strand
tcctttcaaat

SEQ ID NO: 63 - DNA, 11 bp, Artificial Sequence
  3′-sequence to the polymorphic sites on the non-coding strand
tgtggatgcaa

SEQ ID NO: 64 - DNA, 11 bp, Artificial Sequence
  3′-sequence to the polymorphic sites on the non-coding strand
catggctgctt

SEQ ID NO: 65 - DNA, 11 bp, Artificial Sequence
  3′-sequence to the polymorphic sites on the non-coding strand
agggatctccc

SEQ ID NO: 66 - DNA, 11 bp, Artificial Sequence
  3′-sequence to the polymorphic sites on the non-coding strand
taaacaccttt

SEQ ID NO: 67 - DNA, 11 bp, Artificial Sequence
  3′-sequence to the polymorphic sites on the non-coding strand
tgttctttata

SEQ ID NO: 68 - DNA, 11 bp, Artificial Sequence
  3′-sequence to the polymorphic sites on the coding strand
ttgcatccaca

SEQ ID NO: 69 - DNA, 11 bp, Artificial sequence
  3′-sequence to the polymorphic sites on the coding strand
aagcagccatg

SEQ ID NO: 70 - DNA, 11 bp, Artificial Sequence
  3′-sequence to the polymorphic sites on the coding strand
gggagatccct

SEQ ID NO: 71 - DNA, 11 bp, Artificial Sequence
  3′-sequence to the polymorphic sites on the coding strand
aaaggtgttta

SEQ ID NO: 72 - DNA, 11 bp, Artificial Sequence
  3′-sequence to the polymorphic sites on the coding strand
tataaagaaca

SEQ ID NO: 73 - DNA, 29 bp, Artificial Sequence
  Nuclelotide tail complementary to M13 at its 5′-end.
agtcacgacgttgtaaaacgacggccagt

(19) United States
(12) Patent Application Publication
(10) Pub. No.: US 2003/0017469 A1
(43) Pub. Date: Jan. 23, 2003

(54) Detection of CYP3A4 and CYP2C9 polymorphisms

(76) Inventors: Carl Risinger, Uppsala (SE); Maria Kristina Andersson, Uppsala (SE); Tommy Lewander, Uppsala (SE); Erik Olaisson, Bonadsvagen (SE)

Correspondence Address: MORRISON & FOERSTER LLP, 3811 VALLEY CENTRE DRIVE, SUITE 500, SAN DIEGO, CA, 92130-2332, US

(21) Appl. No.: 09/943,115

(22) Filed: Aug. 30, 2001

(30) Foreign Application Priority Data
Aug. 30, 2000 (GB) 0021286.0

(51) Int. Cl. C12Q 1/68; C07H 21/04

(52) U.S. Cl. 435/6; 536/24.3

(57) ABSTRACT

The invention provides oligonucleotide primer pairs, sequence determination oligonucleotides, and kits for amplification and detection of novel single nucleotide polymorphisms in the 5′ flanking regions of the CYP3A4 and CYP2C9 genes.

### DETECTION OF CYP3A4 AND CYP2C9 POLYMORPHISMS

The present invention is directed to methods of preparing biological samples for nucleic acid analysis using oligonucleotide primers suitable for amplification of the genes encoding the drug-metabolizing cytochrome P450 enzymes CYP3A4 and CYP2C19.

### BACKGROUND OF THE INVENTION

Xenobiotics are pharmacologically, endocrinologically, or toxicologically active substances foreign to a biological system. Most xenobiotics, including pharmaceutical agents, are metabolized through two successive reactions. Phase I reactions (functionalization reactions), include oxidation, reduction, and hydrolysis, in which a derivatizable group is added to the original molecule. Functionalization prepares the drug for further metabolism in phase II reactions. During phase II reactions (conjugative reactions, which include glucoronidation, sulfation, methylation and acetylation), the functionalized drug is conjugated with a hydrophilic group. The resulting hydrophilic compounds are inactive and excreted in bile or urine. Thus, metabolism can result in detoxification and excretion of the active substance. Alternatively, an inert xenobiotic may be metabolized to an active compound. For example, a pro-drug may be converted to a biologically active therapeutic or toxin.

The cytochrome P450 (CYP) enzymes are involved in the metabolism of many different xenobiotics. CYPs are a superfamily of heme-containing enzymes, found in eukaryotes (both plants and animals) and prokaryotes, and are responsible for Phase I reactions in the metabolic process. In total, over 500 genes belonging to the CYP superfamily have been described and divided into subfamilies, CYP1-CYP27. In humans, more than 35 genes and 7 pseudogenes have been identified. Members of three CYP gene families, CYP1, CYP2, and CYP3, are responsible for the majority of drug metabolism. The human CYPs which are of greatest clinical relevance for the metabolism of drugs and other xenobiotics are CYP1A2, CYP2A6, CYP2C9, CYP2C19, CYP2D6, CYP2E1 and CYP3A4. The liver is the major site of activity of these enzymes, however CYPs are also expressed in other tissues.

The most important drug-metabolizing CYP enzyme is CYP3A4, which is the major CYP expressed in liver. Expression of the gene encoding CYP3A4 (CYP3A4) is inducible by many commonly used drugs, such as dexamethasone, rifampicin, and clotrimazole. CYP3A4 is estimated to metabolize more than 60% of all drugs in clinical use, including calcium channel blockers such as nifedipine, immunosuppressants such as cyclosporin A, macrolide antibiotics such as erythromycin, and steroid hormones. In addition, CYP3A4 metabolizes some carcinogens, and may be implicated in an individual's susceptibility to such toxins.

The existence of more than one form of the CYP3A4 enzyme is caused by polymorphisms in the gene which encodes the CYP3A4 enzyme (the gene being denoted in italics, as CYP3A4). In fact, almost 20 polymorphisms in the CYP3A4 gene have been described (see http://www.imm.ki.se/cypalleles/ for listing). The distribution of particular CYP3A4 polymorphisms differs among ethnic groups, however, concomitant differences in CYP3A4 activity and responses to drugs which are CYP3A4 substrates remain to be investigated. CYP3A4*1A is the wild type gene, corresponding to the cDNA having GenBank Accession No. A18907 and the genomic DNA having GenBank Accession No. AF280107. A number of mutations in the 5′ untranslated region of CYP3A4 have been described. CYP3A4*1B is an A to G substitution at position −392. CYP3A4*1C is a T to G substitution at position −444. CYP3A4*1D is a C to A substitution at position −62. CYP3A4*1E is a T to A substitution at position −369. CYP3A4*1F is a C to G substitution at −747. The 5′ flanking region of CYP3A4 is set forth in SEQ ID NO: 1 and in FIG. 1.

WO 01/20025 discloses single nucleotide polymorphisms in various exons, introns, and in the 3′ UTR of CYP3A4, as well as oligonucleotides for use in diagnosing and treating abnormal expression and/or function of this gene. WO 00/24926 discloses oligonucleotides for use in detecting an A to G point mutation at position −290 of CYP3A4. WO 99/13106 discloses polymorphisms in CYP3A4, including an A to G substitution at position −392 of the promoter, at the 7^{th} position of the 10 bp NFSE, within oligonucleotides having sequences ACAAGGGCAAGAGAGAGGC (SEQ ID NO:2) and ACAAGGGCAGGAGAGAGGC (SEQ ID NO:3), with polymorphic variants indicated in bold type.

U.S. Pat. No. 6,174,684 and corresponding WO 00/09752 disclose an A to G variant in the nifedipine-specific regulatory element located at positions −287 to −296 of CYP3A4, which is associated with increased risk of prostate cancer and with increased risk of developing leukemia after administration of an epipodophyllotoxin. U.S. Pat. No. 6,174,684 also discloses the oligonucleotides AGGGCAAGAG (SEQ ID NO:4) and AGGGCAGGAG (SEQ ID NO:5), with polymorphic variants indicated in bold type. Rebbeck, et al. (1998) J. Natl. Cancer Inst. 90, 1225-1229 also describes this association between prostate cancer, leukemia, and the A to G mutation.

Kuehl, et al. (2001) Nature Genetics 27, 383-391 discloses mutations at positions −341, −288, and −43 of the CYP3A4 promoter, none of which were associated with altered CYP3A4 activity. Kuehl, et al. also discloses differential distribution of these polymorphisms among Caucasians and African Americans.

A second important CYP enzyme is CYP2C9, which is active in hydroxylation of such drugs as tolbutamide, phenytoin, S-warfarin, diclofenac, ibuprofen, and losarten. The sequence of CYP2C9 is set forth in SEQ ID NO:6. Six variants in CYP2C9 are described on the CYP web site, and another six variant designations are listed without descriptions. The CYP2C9*1 variant is designated as the wild type. Four of the five polymorphic CYP2C9 forms described contain mutations in the coding regions of the gene that result in decreased in vitro activity, and the remaining variant, CYP2C9*6, is a deletion of an A at position 818 which results in a frame shift.

WO00/12757 discloses primer extension assays and kits for detection of the single nucleotide polymorphisms CYP2C9*2 and CYP2C9*3, both of which result in amino acid substitutions.

On the basis of ability of metabolize a marker drug such as nifedipine for CYP3A4 or S-warfarin for CYP2C9, individuals may be characterized as poor metabolizers (PM), intermediate metabolizers (IM), extensive metabolizers (EM) or ultra extensive metabolizers (UEM or UM) for CYP3A4 or CYP2C9 substrates, respectively. Poor metabolizers retain the substrate in their bodies for a relatively long period of time, and are susceptible to toxicity and side effects at “normal” dosages. Ultraextensive metabolizers clear the substrate from their bodies quickly, and require higher than “normal” dosages to achieve a therapeutic effect. Intermediate and extensive metabolizers retain the substrate in their bodies for times between those of PMs and UEMs, and are more likely to respond to “normal” dosages of the drug. However, individuals characterized as IM or EM may differ in drug clearance by as much as 10-fold, and variations in toxicity, side effects, and efficacy for a particular drug may occur among these individuals. However, administration of such drugs to determine an individual's metabolic capacity may in itself be dangerous, exposing the individual to potential toxic side effects.

A need remains for methods of preparing biological samples that contain the 5′ flanking regions of CYP3A4 or CYP2C9, so that this information may be used to predict differential capacities for metabolizing CYP3A4 and CYP2C9 substrates among individuals.

### SUMMARY OF THE INVENTION

The present inventors have discovered a novel single nucleotide polymorphism in the 5′ flanking region of CYP3A4, and six novel polymorphisms in the 5′ flanking region of CYP2C9. Oligonucleotides have been devised for amplification of the polymorphic regions corresponding to these polymorphisms. These oligonucleotides may be used to prepare biological samples for further analysis of the 5′ flanking regions of these genes. The inventors have also devised sequence determination oligonucleotides for use as probes for the novel single nucleotide polymorphisms in CYP3A4 and CYP2C9.

In one embodiment, the invention provides an oligonucleotide primer pair suitable for amplifying a polymorphic region of a 5′ flanking region of a CYP3A4 gene, wherein the polymorphic region corresponds to position 461 of SEQ ID NO:1, which position may also be described as position −644 from the transcription start site of the CYP3A4 gene.

In another embodiment, the invention provides a sequence determination oligonucleotide for detecting a polymorphic site in a 5′ flanking region of a CYP3A4 gene, said oligonucleotide being complementary to the polymorphic region corresponding to position 461 of SEQ ID NO:1.

In another embodiment, the invention provides a kit for amplification and/or detection of a polymorphic region of the 5′ flanking region of a CYP3A4 gene, said kit comprising at least one oligonucleotide primer pair capable of amplifying the region corresponding to position 461 of SEQ ID NO:1.

In another embodiment, the invention provides an oligonucleotide primer pair suitable for amplifying a polymorphic region of a 5′ flanking region of a CYP2C9 gene, wherein the polymorphic region corresponds to position 957 of SEQ ID NO:6; position 1049 of SEQ ID NO:6; position 1164 of SEQ ID NO:6; position 1526 of SEQ ID NO:6; position 1661 of SEQ ID NO:6; and position 1662 of SEQ ID NO:6. Position 957 of SEQ ID NO:6 may also be described as position −1189 from the transcription start site of the CYP3C9 gene; position 1049 of SEQ ID NO:6 may also be described as position −1097 from the transcription start site; position 1164 of SEQ ID NO:6 may also be described as position −982 from the transcription start site; position 1526 of SEQ ID NO:6 may also be described as position −620 from the transcription start site; position 1661 of SEQ ID NO:6 may also be described as position −485 from the transcription start site; and position 1662 of SEQ ID NO:6 may also be described as position −484 from the transcription start site.

In yet another embodiment, the invention provides a sequence determination oligonucleotide for detecting a polymorphic site in a 5′ flanking region of a CYP2C9 gene, said oligonucleotide comprising a sequence selected from the group consisting of an oligonucleotide complementary to the polymorphic region corresponding to position 957 of SEQ ID NO:6; an oligonucleotide complementary to the polymorphic region corresponding to position 1049 of SEQ ID NO:6; an oligonucleotide complementary to the polymorphic region corresponding to position 1164 of SEQ ID NO:6; an oligonucleotide complementary to the polymorphic region corresponding to position 1526 of SEQ ID NO:6; an oligonucleotide complementary to the polymorphic region corresponding to position 1661 of SEQ ID NO:6; and an oligonucleotide complementary to the polymorphic region corresponding to position 1662 of SEQ ID NO:6.

In another embodiment, the invention provides a kit for amplification and/or detection of a polymorphic region corresponding to at least one polymorphic region in the 5′ flanking region of the CYP2C9 gene, said region being selected from the group consisting of position 957 of SEQ ID NO:6; position 1049 of SEQ ID NO:6; position 1164 of SEQ ID NO:6; position 1526 of SEQ ID NO:6; position 1661 of SEQ ID NO:6; and position 1662 of SEQ ID NO:6.

### BRIEF DESCRIPTION OF THE DRAWINGS

FIG. 1 shows the sequence of the 5′ flanking region of the CYP3A4 gene as set forth in SEQ ID NO: 1, with the novel polymorphic site underlined and highlighted in bold.

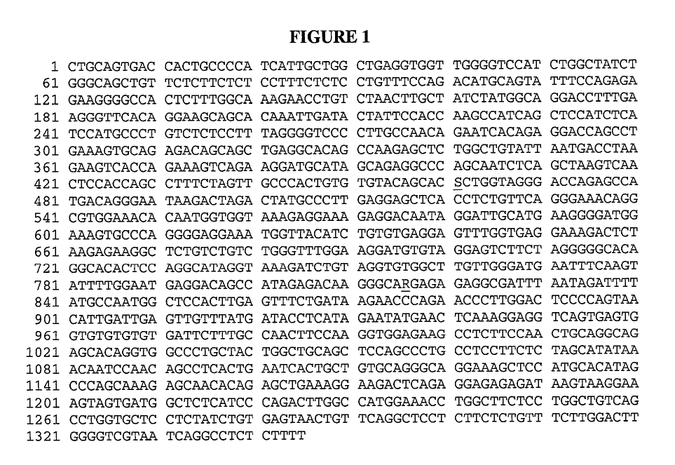

FIG. 2 shows the sequence of the 5′ flanking region of the CYP2C9 gene as set forth in SEQ ID NO:6, with the novel polymorphic sites underlined and highlighted in bold.

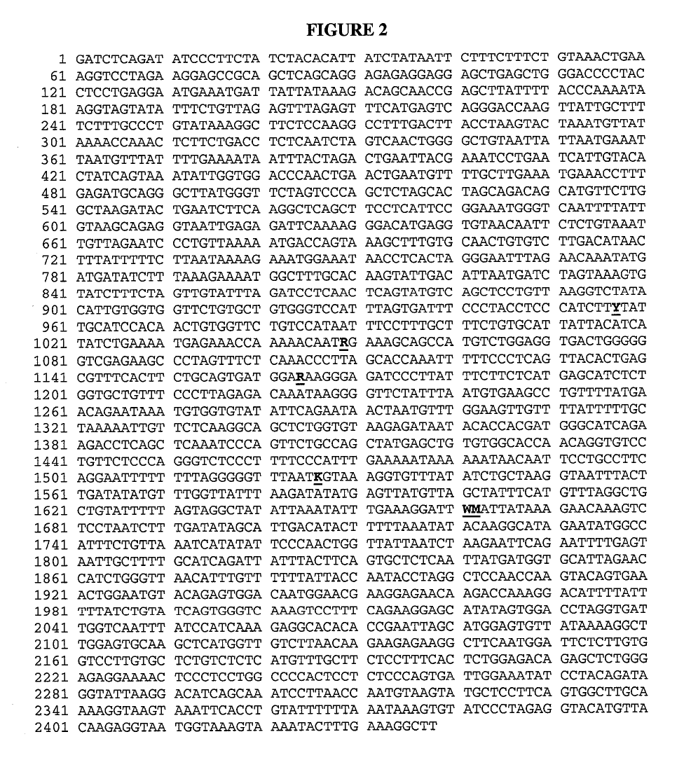

### DETAILED DESCRIPTION OF THE INVENTION

The U.S. patents and publications referenced herein are hereby incorporated by reference.

For the purposes of the invention, certain terms are defined as follows. “Gene” is defined as the genomic sequence of the CYP2C19 gene. “Oligonucleotide” means a nucleic acid molecule preferably comprising from about 8 to about 50 covalently linked nucleotides. More preferably, an oligonucleotide of the invention comprises from about 8 to about 35 nucleotides. Most preferably, an oligonucleotide of the invention comprises from about 10 to about 25 nucleotides. In accordance with the invention, the nucleotides within an oligonucleotide may be analogs or derivatives of naturally occurring nucleotides, so long as oligonucleotides containing such analogs or derivatives retain the ability to hybridize specifically within the polymorphic region containing the targeted polymorphism. Analogs and derivatives of naturally occurring oligonucleotides within the scope of the present invention are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and the like. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372, and the like. The term “oligonucleotides” as defined herein also includes compounds which comprise the specific oligonucleotides disclosed herein, covalently linked to a second moiety. The second moiety may be an additional nucleotide sequence, for example, a tail sequence such as a polyadenosine tail or an adaptor sequence, for example, the phage M13 universal tail sequence, and the like. Alternatively, the second moiety may be a non-nucleotidic moiety, for example, a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the specific oligonucleotide, so long as the oligonucleotide retains its ability to hybridize to the polymorphic regions described herein.

A polymorphic region as defined herein is a portion of a genetic locus that is characterized by at least one polymorphic site. A genetic locus is a location on a chromosome which is associated with a gene, a physical feature, or a phenotypic trait. A polymorphic site is a position within a genetic locus at which at least two alternative sequences have been observed in a population. A polymorphic region as defined herein is said to “correspond to” a polymorphic site, that is, the region may be adjacent to the polymorphic site on the 5′ side of the site or on the 3′ side of the site, or alternatively may contain the polymorphic site. A polymorphic region includes both the sense and antisense strands of the nucleic acid comprising the polymorphic site, and may have a length of from about 100 to about 5000 base pairs. For example, a polymorphic region may be all or a portion of a regulatory region such as a promoter, 5′ UTR, 3′ UTR, an intron, an exon, or the like. A polymorphic or allelic variant is a genomic DNA, cDNA, mRNA or polypeptide having a nucleotide or amino acid sequence that comprises a polymorphism. A polymorphism is a sequence variation observed at a polymorphic site, including nucleotide substitutions (single nucleotide polymorphisms or SNPs), insertions, deletions, and microsatellites. Polymorphisms may or may not result in detectable differences in gene expression, protein structure, or protein function. Preferably, a polymorphic region of the present invention has a length of about 1000 base pairs. More preferably, a polymorphic region of the invention has a length of about 500 base pairs. Most preferably, a polymorphic region of the invention has a length of about 200 base pairs.

A haplotype as defined herein is a representation of the combination of polymorphic variants in a defined region within a genetic locus on one of the chromosomes in a chromosome pair. A genotype as used herein is a representation of the polymorphic variants present at a polymorphic site.

The PCR primer pairs of the invention are capable of amplifying the polymorphic region corresponding to position 461 of SEQ ID NO: 1, or any of the polymorphic regions corresponding to position 957 of SEQ ID NO:6; position 1049 of SEQ ID NO:6; position 1164 of SEQ ID NO:6; position 1526 of SEQ ID NO:6; position 1661 of SEQ ID NO:6; and position 1662 of SEQ ID NO:6. Specific oligonucleotide primer pairs of the invention, for amplifying position 461 of SEQ ID NO:1, may comprise sequences selected from the group consisting of SEQ ID NO:7 and SEQ ID NO:8; and SEQ ID NO;9 and SEQ ID NO: 10. For amplifying only position 957 of SEQ ID NO:6, an oligonucleotide primer pair comprising the sequences set forth in SEQ ID NO: 19 and SEQ ID NO:20 may be used. Alternatively, positions 957 and 1049 of SEQ ID NO:6 may be amplified using an oligonucleotide primer pair comprising the sequences set forth in SEQ ID NO:21 and SEQ ID NO:22; or positions 957,1049, and 1164 may be amplified using an oligonucleotide primer pair comprising the sequences set forth in SEQ ID NO:23 and SEQ ID NO:24. Position 1164 of SEQ ID NO:6 may also be amplified using an oligonucleotide primer pair comprising the sequences set forth in SEQ ID NO:25 and SEQ ID NO:26. Positions 1526, 1661, and 1662 of SEQ ID NO:6 may be amplified using an oligonucleotide primer pair comprising the sequences set forth in SEQ ID NO:27 and SEQ ID NO:28. Positions 1661 and 1662 of SEQ ID NO:6 may be amplified using an oligonucleotide primer pair selected from the group consisting of an oligonucleotide primer pair comprising the sequences set forth in SEQ ID NO:29 and SEQ ID NO:30 and an oligonucleotide primer pair comprising the sequences set forth in SEQ ID NO:31 and SEQ ID NO:32.

Each of the PCR primer pairs of the invention may be used in any PCR method. For example, a PCR primer pair of the invention may be used in the methods disclosed in U.S. Pat. Nos. 4,683,195; 4,683,202, 4,965,188; 5,656,493; 5,998,143; 6,140,054; WO 01/27327; WO 01/27329; and the like. The PCR pairs of the invention may also be used in any of the commercially available machines that perform PCR, such as any of the GeneAmp® Systems available from Applied Biosystems.

The oligonucleotides of the invention may be used to determine the sequence of the polymorphic regions of SEQ ID NO: 1 or SEQ ID NO:6 as defined herein. In one embodiment, an oligonucleotide of the invention comprises a sequence selected from the group consisting of SEQ ID NO:11; SEQ ID NO:12; SEQ ID NO:13; SEQ ID NO:14; SEQ ID NO:15; SEQ ID NO: 16; SEQ ID NO:17; and SEQ ID NO:18, for determining the sequence of the novel polymorphic region of CYP3A4 corresponding to position 461 of SEQ ID NO:1. In another embodiment, for determining the sequence of the polymorphic region of CYP2C9 corresponding to position 957 of SEQ ID NO:6, an oligonucleotide of the invention comprises a sequence selected from the group consisting of SEQ ID NO:33; SEQ ID NO:34; SEQ ID NO:43; SEQ ID NO:44; SEQ ID NO:53; SEQ ID NO:58; SEQ ID NO:63; and SEQ ID NO:68. In another embodiment, for determining the sequence of the polymorphic region of CYP2C9 corresponding to position 1049 of SEQ ID NO:6, an oligonucleotide of the invention comprises a sequence selected from the group consisting of SEQ ID NO:35; SEQ ID NO:36; SEQ ID NO:45; SEQ ID NO:46; SEQ ID NO:54; SEQ ID NO:59; SEQ ID NO:64; and SEQ ID NO:69. In another embodiment, for determining the sequence of the polymorphic region of CYP2C9 corresponding to position 1164 of SEQ ID NO:6, an oligonucleotide of the invention comprises a sequence selected from the group consisting of SEQ ID NO:37; SEQ ID NO:38; SEQ ID NO:45; SEQ ID NO:48; SEQ ID NO:55; SEQ ID) NO:60; SEQ ID) NO:65; and SEQ ID NO:70. In another embodiment, for determining the sequence of the polymorphic region of CYP2C9 corresponding to position 1526 of SEQ ID NO:6, an oligonucleotide of the invention comprises a sequence selected from the group consisting of SEQ ID NO:39; SEQ ID NO:40; SEQ ID NO:49; SEQ ID NO:50; SEQ if) NO:56; SEQ ID NO:61; SEQ ID NO:66; and SEQ ID NO:71. In another embodiment, for determining the sequences of the polymorphic region of CYP2C9 corresponding to either of positions 1661 or 1662 of SEQ ID NO:6, an oligonucleotide of the invention comprises a sequence selected from the group consisting of SEQ ID NO:41; SEQ ID NO:42; SEQ ID NO:51; SEQ ID NO:52; SEQ ID NO:57; SEQ ID NO:62; SEQ ID NO:67; and SEQ ID NO:72.

Those of ordinary skill will recognize that oligonucleotides complementary to the polymorphic regions described herein must be capable of hybridizing to the polymorphic regions under conditions of stringency such as those employed in primer extension-based sequence determination methods, restriction site analysis, nucleic acid amplification methods, ligase-based sequencing methods, methods based on enzymatic detection of mismatches, microarray-based sequence determination methods, and the like. The oligonucleotides of the invention may be synthesized using known methods and machines, such as the ABI™3900 High Throughput DNA Synthesizer and the Expedite™8909 Nucleic Acid Synthesizer, both of which are available from Applied Biosystems (Foster City, Calif.).

The oligonucleotides of the invention may be used, without limitation, as in situ hybridization probes or as components of diagnostic assays. Numerous oligonucleotide-based diagnostic assays are known. For example, primer extension-based nucleic acid sequence detection methods are disclosed in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; WO 01/20039; and the like. Primer extension-based nucleic acid sequence detection methods using mass spectrometry are described in U.S. Pat. Nos. 5,547,835; 5,605,798; 5,691,141; 5,849,542; 5,869,242; 5,928,906; 6,043,031; 6,194,144, and the like. The oligonucleotides of the invention are also suitable for use in ligase-based sequence determination methods such as those disclosed in U.S. Pat. Nos. 5,679,524 and 5,952,174, WO 01/27326, and the like. The oligonucleotides of the invention may be used as probes in sequence determination methods based on mismatches, such as the methods described in U.S. Pat. Nos. 5,851,770; 5,958,692; 6,110,684; 6,183,958; and the like. In addition, the oligonucleotides of the invention may be used in hybridization-based diagnostic assays such as those described in U.S. Pat. Nos. 5,891,625; 6,013,499; and the like.

The oligonucleotides of the invention may also be used as components of a diagnostic microarray. Methods of making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; WO 01/29259; and the like.

The invention is also embodied in a kit comprising at least one oligonucleotide primer pair of the invention. When the kit is used for amplification and detection of CYP3A4 polymorphisms, it will comprise an oligonucleotide primer pair suitable for amplification of the polymorphic region corresponding to position 461 of SEQ ID NO:1.

Specific primer pairs in this embodiment are selected from the group consisting of SEQ ID NO:7 and SEQ ID NO:8; and SEQ ID NO;9 and SEQ ID NO:10. This embodiment of the kit of the invention may optionally comprise a sequence determination oligonucleotide selected from the group consisting of SEQ ID NO: 11; SEQ ID NO: 12; SEQ ID NO:13; SEQ ID NO: 14; SEQ ID NO:15; SEQ ID NO:16; SEQ ID NO:17; and SEQ ID NO:18.

When the kit of the invention is used for amplification and detection of polymorphisms in the 5′ flanking region of CYP2C9, it will comprise at least one oligonucleotide primer pair, wherein the primer pair is capable of amplifying a polymorphic region selected from the group consisting of the polymorphic region corresponding to position 957 of SEQ ID NO:6; the polymorphic region corresponding to position 1049 of SEQ ID NO:6; the polymorphic region corresponding to position 1164 of SEQ ID NO:6; the polymorphic region corresponding to position 1526 of SEQ ID NO:6; the polymorphic region corresponding to position 1661 of SEQ ID NO:6; and the polymorphic region corresponding to position 1662 of SEQ ID NO:6. This embodiment may optionally further comprise a sequence determination oligonucleotide for detecting a polymorphic variant at any or all of the polymorphic sites corresponding to positions 957, 1049, 1164, 1526, 1661 and 1662 of SEQ ID NO:6.

The kit of the invention may also comprise a polymerizing agent, for example, a thermostable nucleic acid polymerase such as those disclosed in U.S. Pat. Nos. 4,889,818; 6,077,664, and the like. The kit of the invention may also comprise chain elongating nucleotides, such as dATP, dTTP, dGTP, dCTP, and dITP, including analogs of dATP, dTTP, dGTP, dCTP and dITP, so long as such analogs are substrates for a thermostable nucleic acid polymerase and can be incorporated into a growing nucleic acid chain. The kit of the invention may also include chain terminating nucleotides such as ddATP, ddTTP, ddGTP, ddCTP, and the like. In a preferred embodiment, the kit of the invention comprises at least two oligonucleotide primer pairs, a polymerizing agent, chain elongating nucleotides, at least two sequence determination oligonucleotides and at least one chain terminating nucleotide. The kit of the invention may optionally include buffers, vials, microtiter plates, and instructions for use.

The examples set forth below are provided as illustration and are not intended to limit the scope and spirit of the invention as specifically embodied therein.

### EXAMPLE 1

### IDENTIFICATION OF CYP3A4 POLYMORPHISM

The study was performed in accordance with the principles stated in the Declaration of Helsinki as reviewed in Tokyo 1975 and Venice 1983, Hong Kong 1989 and Somerset West 1996. Ten samples (Swedish Caucasians) were selected and used for identification of polymorphisms in the 5′ flanking region of CYP3A4.

White blood cells isolated from a blood sample drawn from the brachial vein serve as the source of the genomic DNA for the analyses. The DNA was extracted by guanidine thiocyanate method or Q1Aamp Blood Kit (QIAGEN, Venlo, The Netherlands). The genes included in the study were amplified by PCR and the DNA sequences were determined by full sequencing. All genetic analyses were performed according to Good Laboratory Practice and Standard Operating Procedures. Case Report Forms were designed and used for clinical and genetic data collection. Data was entered and stored in a relational database at Gemini Genomics AB, Uppsala. To secure consistency between the Case Report Forms and the database, data was checked either by double data entry or proofreading. After a Clean File was declared the database was protected against changes. By using the program Stat/Transfer™ the database was transferred to SAS data sets. The SAS™ system was used for tabulations and statistical evaluations. Genotypes were also correlated against the metabolic ratio.

PCR-fragments were amplified with TaqGOLD polymerase (Applied Biosystems) using Robocycler (Stratagene) or GeneAmp PCR system 9700 (Applied Biosystems). Preferentially, the amplified fragments were 300-400 bp, and the region to be read did not exceed 300 bp. PCR reactions were carried out according to the basic protocol set forth in Table 1, with modifications as indicated in Table 2 for specific primer pairs, which are shown in Table 3. For the GeneAmp PCR 9700 machine the profile used was 10 minutes at 95°, 40×(45 seconds at 90°, 45 seconds at 60°, 45 seconds at 72°), 5 minutes at 72° and 22° until removed.

TABLE 1

| Solution | Stock Concentration | PCR (μl) |
|---|---|---|
| H₂O | | 33.2 |
| PCR buffer | 10x | 5.0 |
| MgCl₂ | 25 mM | 2.0 |
| dNTP | 2.5 mM | 2.5 |
| primer 1 | 10 μM | 1.0 |
| primer 2 | 10 μM | 1.0 |
| Taq-gold | 5 μ/μl | 0.3 |
| polymerase | | |
| DNA samples | 2 ng/μl | 5.0 |
| TOTAL | | 50.0 |

TABLE 2

| SEQ ID | Polymorphic | Modification from basic | |
|---|---|---|---|
| NO:s | Site | protocol (Table 1) | Detection method |
| 7, 8 | 461 | 62° annealing temperature | Full sequencing |
| 9, 10 | 461 | 3 μl MgCl_{2,} 58° annealing | Full sequencing |
| | | temperature, 50 cycles | |

TABLE 3

| Polymorphic | | |
|---|---|---|
| Site | Primer Pair | |
| 461 | SEQ ID NO:7 | CCAGCCTGAAAGTGCAGAGA |
| | SEQ ID NO:8 | TCTTAGAGTCTTTCCTCACCAAACT |
| 461 | SEQ ID NO:9 | CATGCCCTGTCTCTCCTTTA |
| | SEQ ID NO:10 | CCATCCCCTTCATGCAATC |

The optimized condition specified in Table 2 were required to distinguish CYP3A4 from the closely related gene-family members CYP3A5, and CYP3A7. Use of the basic protocol will lead to problems when amplifying CYP3A4-specific amplicons of 300-400 bp containing the polymorphisms of interest, unless a nested PCR approach is carried out. The nested PCR approach was not used because of the high risk of contamination when using a nested PCR approach and the high risk of typing errors as a consequence. The modifications shown in Table 2 were optimized and reaction parameters were balanced in such a way that nested PCR was avoided.

For full sequencing, one of the PCR-primers in a primer pair was designed for sequencing by addition of a 29 nucleotide tail complementary to M13 at its 5′-end, namely the nucleotides AGTCACGACGTTGTAAAACGACGGCCAGT. Thus, the entire PCR-product was sequenced from the tailed PCR-primer.

The additional oligonucleotides set forth in Tables 4 through 7 were identified as being suitable for detection of the SNP at positions 461 of the 5′ flanking region of the CYP3A4 gene as depicted in SEQ ID NO: 1.

Table 4 sets forth oligonucleotides representing the coding (sense) strand complementary to the polymorphic region corresponding to the novel polymorphism found in the study population. The underlined letter indicates polymorphic position in the sequence context. All sequences are shown in 5′ to 3′ direction.

TABLE 4

| | | |
|---|---|---|
| Polymorphic | | |
| Site | Sequence | Note |

| | | | |
|---|---|---|---|
| 461 | SEQ ID NO:11: | AGCACCCTGGT | C variant |
| | SEQ ID NO:12: | AGCACGCTGGT | G variant |

Table 5 sets forth oligonucleotides representing the non-coding (anti-sense) strand complementary to the polymorphic region corresponding to the novel polymorphism found in the study population. The underlined letter indicates polymorphic position in the sequence context. All sequences are shown in 5′ to 3′ direction.

TABLE 5

| Poly- | | | |
|---|---|---|---|
| mor- | | | |
| phic | | | |
| Site | Sequence | | Note |
| 461 | SEQ ID NO:13: | ACCAGGGTGCT | Antisense G variant |
| | SEQ ID NO:14: | ACCAGCGTGCT | Antisense C variant |

The sequences of Table 6 represent the 5′-sequence to the novel polymorphic site on the coding (sense) strand (SEQ ID NO: 15) and non-coding (anti-sense) strand (SEQ ID NO:s 16). All sequences are shown in 5′ to 3′ direction.

TABLE 6

| Polymorphic | | | |
|---|---|---|---|
| Site | Sequence | | Note |
| 461 | SEQ ID NO:15: | GTGTGTACAGC | Sense 5′ |
| | SEQ ID NO:16: | GCTGTACACAC | Antisense 5′ |

The sequences of Table 7 represent the 3′-sequence to the novel polymorphic site on the non-coding (anti-sense) strand (SEQ ID NO: 17) and the coding (sense) strand (SEQ ID NO:18). All sequences are shown in 5′ to 3′ direction.

TABLE 9

| Polymorphic | | | |
|---|---|---|---|
| Site | Sequence | | Note |
| 461 | SEQ ID NO:17: | TGGTCCCTACC | Antisense 3′ |
| | SEQ ID NO:18: | GGTAGGGACCA | Sense 3′ |

### EXAMPLE 2

### IDENTIFICATION OF CYP2C9 POLYMORPHISMS

The study was performed in accordance with the principles stated in the Declaration of Helsinki as reviewed in Tokyo 1975 and Venice 1983, Hong Kong 1989 and Somerset West 1996. Ten samples (Swedish Caucasians) were selected and used for identification of polymorphisms in the 5′ flanking region of CYP2C9.

White blood cells isolated from a blood sample drawn from the brachial vein serve as the source of the genomic DNA for the analyses. The DNA is extracted by guanidine thiocyanate method or Q1Aamp Blood Kit (QIAGEN, Venlo, The Netherlands). The genes included in the study were amplified by PCR and the DNA sequences were determined by full sequencing. All genetic analyses were performed according to Good Laboratory Practice and Standard Operating Procedures. Case Report Forms were designed and used for clinical and genetic data collection. Data was entered and stored in a relational database at Gemini Genomics AB, Uppsala. To secure consistency between the Case Report Forms and the database, data was checked either by double data entry or proofreading. After a Clean File was declared the database was protected against changes. By using the program Stat/Transfer™ the database was transferred to SAS data sets. The SAS™ system was used for tabulations and statistical evaluations. Genotypes were also correlated against the metabolic ratio.

PCR-fragments were amplified with TaqGOLD polymerase (Applied Biosystems) using Robocycler (Stratagene) or GeneAmp PCR system 9700 (Applied Biosystems). Preferentially, the amplified fragments were 300-400 bp, and the region to be read did not exceed 300 bp. PCR reactions were carried out according to the basic protocol set forth in Table 10, with modifications as indicated in Table 11 for specific primer pairs, which are shown in Table 12. For the GeneAmp PCR 9700 machine the profile used was 10 minutes at 95°, 40×(45 seconds at 90°, 45 seconds at 60°, 45 seconds at 72°), 5 minutes at 72° and 22° until removed.

TABLE 10

| Solution | Stock Concentration | PCR (μl) |
|---|---|---|
| H₂O | | 33.2 |
| PCR buffer | 10x | 5.0 |
| MgCl₂ | 25 mM | 2.0 |
| dNTP | 2.5 mM | 2.5 |
| primer 1 | 10 μM | 1.0 |
| primer 2 | 10 μM | 1.0 |
| Taq-gold | 5 μ/μl | 0.3 |
| polymerase | | |
| DNA samples | 2 ng/μl | 5.0 |
| TOTAL | | 50.0 |

TABLE 11

| SEQ ID | Polymorphic | Modification from basic | |
|---|---|---|---|
| NO:s | Site | protocol (Table 10) | Detection method |
| 19, 20 | 957 | 58° annealing temperature | Full sequencing |
| 21, 22 | 957 & 1049 | 3 μl MgCl_{2,} 62° annealing | Full sequencing |
| | | temperature | |
| 23, 24 | 957, | 58° annealing temperature | Full sequencing |
| | 1049 & 1164 | | |
| 25, 26 | 1164 | 3 μl MgCl₂, 62° annealing | Full sequencing |
| | | temperature, 50 cycles | |
| 27, 28 | 1526, | | Full sequencing |
| | 1661 & 1662 | | |
| 29, 30 | 1661 & 1662 | 3 μl MgCl₂, 62° annealing | Full sequencing |
| | | temperature, 50 cycles | |
| 31, 32 | 1661 & 1662 | | Full sequencing |

TABLE 12

| Polymorphic | | |
|---|---|---|
| Site | Primer Pair | |
| 957 | SEQ ID NO:19 | CACTAGGGAATTTAGAACAAATATG |
| | SEQ ID NO:20 | GCACAGAAAGCAAAGGAAATTAT |
| 957 & 1049 | SEQ ID NO:21 | TGTATTTAGATCCTCAACTCAGTATGT |
| | SEQ ID NO:22 | GGATCTCCCTTCTCCATCACT |
| 957, 1049 & 1164 | SEQ ID NO:23 | GGTCCATTTAGTGATTTCCCTAC |
| | SEQ ID NO:24 | ATACACCACATTTATTCTGTTCATA |
| 1164 | SEQ ID NO:25 | CCAAATTTTTCCCTCAGTTACA |
| | SEQ ID NO:26 | TTGGTGCCACACAGCTCATA |
| 1526, 1661 & 1662 | SEQ ID NO:27 | GCCTTCAGGAATTTTTTTTA |
| | SEQ ID NO:28 | CCAGTTGGGAATATATGATTTAACA |
| 1661 & 1662 | SEQ ID NO:29 | GCTGCTGTATTTTTAGTAGGCTATA |
| | SEQ ID NO:30 | CGTTCCATTGTCCACTCTGTAC |
| 1661 & 1662 | SEQ ID NO:31 | TCAAGGCAGCTCTGGTGTAA |
| | SEQ ID NO:32 | AGTTGGGAATATATGATTTAACAGA |

The optimized condition specified in Table 11 were required to distinguish CYP2C9 from the closely related gene-family members CYP2C8, CYP2C18 and CYP2C19. Use of the basic protocol will lead to problems when amplifying CYP2C9-specific amplicons of 300-400 bp containing the polymorphisms of interest, unless a nested PCR approach is carried out. The nested PCR approach was not used because of the high risk of contamination when using a nested PCR approach and the high risk of typing errors as a consequence. The modifications shown in Table 11 were optimized and reaction parameters were balanced in such a way that nested PCR was avoided.

For full sequencing, one of the PCR-primers in a primer pair was designed for sequencing by addition of a 29 nucleotide tail complementary to M13 at its 5′-end, namely the nucleotides AGTCACGACGTTGTAAAACGACGGCCAGT. Thus, the entire PCR-product was sequenced from the tailed PCR-primer. The additional oligonucleotides set forth in Tables 13 through 16 were identified as being suitable for detection of the SNPs at positions 957, 1049, 1164, 1526, 1661 and/or 1662 of the 5′ flanking region of the CYP2C9 gene as depicted in SEQ ID NO:6.

Table 13 sets forth oligonucleotides representing the coding (sense) strand complementary to the polymorphic region corresponding to the polymorphisms found in the study population. The underlined letter indicates polymorphic position in the sequence context. All sequences are shown in 5′ to 3′ direction.

TABLE 13

| Polymorphic | | | |
|---|---|---|---|
| Site | Sequence | | Note |
| 957 | SEQ ID NO:33: | ATCTTCTATTG | C variant |
| | SEQ ID NO:34: | ATCTTTTATTG | T Variant |
| 1049 | SEQ ID NO:35: | ACAATAGAAAG | A variant |
| | SEQ ID NO:36: | ACAATGGAAAG | G variant |
| 1164 | SEQ ID NO:37: | ATGGAGAAGGG | G variant |
| | SEQ ID NO:38: | ATGGAAAAGGG | A variant |
| 1526 | SEQ ID NO:39: | TTAATGGTAAA | G variant |
| | SEQ ID NO:40: | TTAATTGTAAA | T variant |
| 1661 & 1662 | SEQ ID NO:41: | GGATTTCATTAT | TC variants |
| | SEQ ID NO:42: | GGATTAAATTAT | AA variants |

Table 14 sets forth oligonucleotides representing the non-coding (anti-sense) strand complementary to the polymorphic region corresponding to the polymorphisms found in the study population. The underlined letter indicates polymorphic position in the sequence context. All sequences are shown in 5′ to 3′ direction.

TABLE 14

| Poly- | | | |
|---|---|---|---|
| mor- | | | |
| phic | | | |
| Site | Sequence | | Note |
| 957 | SEQ ID NO:43: | CAATAGAAGAT | Antisense G variant |
| | SEQ ID NO:44: | CAATAAAAGAT | Antisense A variant |
| 1049 | SEQ ID NO:45: | CTTTCTATTGT | Antisense T variant |
| | SEQ ID NO:46: | CTTTCCATTGT | Antisense C variant |
| 1164 | SEQ ID NO:47: | CCCTTCTCCAT | Antisense C variant |
| | SEQ ID NO:48: | CCCTTTTCCAT | Antisense T variant |
| 1526 | SEQ ID NO:49: | TTTACCATTAA | Antisense C variant |
| | SEQ ID NO:50: | TTTACAATTAA | Antisense A variant |
| 1661 & | SEQ ID NO:51: | ATAATGAAATCC | Antisense GA variants |
| 1662 | SEQ ID NO:52: | ATAATTTAATCC | Antisense TT variant |

The sequences of Table 15 represent the 5′-sequence to the polymorphic sites on the coding (sense) strand (SEQ ID NO:s 53-57) and non-coding (anti-sense) strand (SEQ ID NO:s 58-67). All sequences are shown in 5′ to 3′ direction.

TABLE 15

| Polymorphic | | | |
|---|---|---|---|
| Site | Sequence | | Note |
| 957 | SEQ ID NO:53: | TACCTCCCATC | Sense 5′ |
| | SEQ ID NO:58: | GATGGGAGGTA | Antisense 5′ |
| 1049 | SEQ ID NO:54: | AACCAAAAACA | Sense 5′ |
| | SEQ ID NO:59: | TGTTTTTGGTT | Antisense 5′ |
| 1164 | SEQ ID NO:55: | CTGCAGTGATG | Sense 5′ |
| | SEQ ID NO:60: | CATCACTGCAG | Antisense 5′ |
| 1526 | SEQ ID NO:56: | TAGGGGGTTTA | Sense 5′ |
| | SEQ ID NO:61: | TAAACCCCCTA | Antisense 5′ |
| 1661 & 1662 | SEQ ID NO:57: | ATTTGAAAGGA | Sense 5′ |
| | SEQ ID NO:62: | TCCTTTCAAAT | Antisense 5′ |

The sequences of Table 16 represent the 3′-sequence to the polymorphic sites on the non-coding (anti-sense) strand (SEQ ID NO:s 68-72) and the coding (sense) strand (SEQ ID NO:s 73-77). All sequences are shown in 5′ to 3′ direction.

TABLE 16

| Polymorphic | | | |
|---|---|---|---|
| Site | Sequence | | Note |
| 957 | SEQ ID NO:63: | TGTGGATGCAA | Antisense 3′ |
| | SEQ ID NO:68: | TTGCATCCACA | Sense 3′ |
| 1049 | SEQ ID NO:64: | CATGGCTGCTT | Antisense 3′ |
| | SEQ ID NO:69: | AAGCAGCCATG | Sense 3′ |
| 1164 | SEQ ID NO:65: | AGGGATCTCCC | Antisense 3′ |
| | SEQ ID NO:70: | GGGAGATCCCT | Sense 3′ |
| 1526 | SEQ ID NO:66: | TAAACACCTTT | Antisense 3′ |
| | SEQ ID NO:71: | AAAGGTGTTTA | Sense 3′ |
| 1661 & 1662 | SEQ ID NO:67: | TGTTCTTTATA | Antisense 3′ |
| | SEQ ID NO:72: | TATAAAGAACA | Sense 3′ |

1. An oligonucleotide primer pair suitable for amplifying a polymorphic region of a 5′ flanking region of a CYP3A4 gene, wherein the polymorphic region corresponds to position 816 of SEQ ID NO:1.

2. The primer pair of claim 1, having sequences selected from the group consisting of SEQ ID NO:7 and SEQ ID NO:8 and SEQ ID NO:9 and SEQ ID NO:10.

3. A sequence determination oligonucleotide for detecting a polymorphic site in a 5′ flanking region of a CYP3A4 gene, said oligonucleotide being complementary to the polymorphic region corresponding to position 461 of SEQ ID NO:1.

4. The oligonucleotide of claim 3, comprising a sequence selected from the group consisting of SEQ ID NO:11; SEQ ID NO:12; SEQ ID NO:13; SEQ ID NO: 14; SEQ ID NO:15; SEQ ID NO:16; SEQ ID NO: 17; and SEQ ID NO:18.

5. A kit comprising at least one oligonucleotide primer pair capable of amplifying the region corresponding to position 461 of SEQ ID NO:1.

6. The kit of claim 5, wherein the primer pair comprises sequences selected from the group consisting of SEQ ID NO: 7 and SEQ ID NO:8 and SEQ ID NO:9 and SEQ ID no: 10.

7. The kit of claim 5, further comprising a sequence determination oligonucleotide complementary to the polymorphic region corresponding to position 461 of SEQ ID NO:1.

8. The kit of claim 7, wherein the oligonucleotide comprises a sequence selected from the group consisting of SEQ ID NO:11; SEQ ID NO:12; SEQ ID NO: 13; SEQ ID NO:14; SEQ ID NO:15;SEQ ID NO:16; SEQ ID NO:17; and SEQ ID NO:18.

9. An oligonucleotide primer pair suitable for amplifying a polymorphic region of a 5′ flanking region of a CYP2C9 gene, wherein the polymorphic region corresponds to position 957 of SEQ ID NO:6; position 1049 of SEQ ID NO:6; position 1164 of SEQ ID NO:6; position 1526 of SEQ ID NO:6; position 1661 of SEQ ID NO:6; and position 1662 of SEQ ID NO:6.

10. The primer pair of claim 9, having a sequence selected from the group consisting of SEQ ID NO: 19 and SEQ ID NO:20; SEQ ID NO:21 and SEQ ID NO:22; SEQ ID NO:23 and SEQ ID NO:24; SEQ ID NO:25 and SEQ ID NO:26; SEQ ID NO:27 and SEQ ID NO:28; SEQ ID NO:29 and SEQ ID NO:30; and SEQ ID NO:31 and SEQ ID NO:32.

11. A sequence determination oligonucleotide for detecting a polymorphic site in a 5′ flanking region of a CYP2C9 gene, said oligonucleotide comprising a sequence selected from the group consisting of an oligonucleotide complementary to the polymorphic region corresponding to position 957 of SEQ ID NO:6; an oligonucleotide complementary to the polymorphic region corresponding to position 1049 of SEQ ID NO:6; an oligonucleotide complementary to the polymorphic region corresponding to position 1164 of SEQ ID NO:6; an oligonucleotide complementary to the polymorphic region corresponding to position 1526 of SEQ ID NO:6; an oligonucleotide complementary to the polymorphic region corresponding to position 1661 of SEQ ID NO:6; and an oligonucleotide complementary to the polymorphic region corresponding to position 1662 of SEQ ID NO:6.

12. The oligonucleotide of claim 11, comprising a sequence selected from the group consisting of SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO:52; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; and SEQ ID NO:68.

13. A kit comprising at least one oligonucleotide primer pair, wherein the primer pair is capable of amplifying a polymorphic region selected from the group consisting of the polymorphic region corresponding to position 957 of SEQ ID NO:6; the polymorphic region corresponding to position 1049 of SEQ ID NO:6; the polymorphic region corresponding to position 1164 of SEQ ID NO:6; the polymorphic region corresponding to position 1526 of SEQ ID NO:6; the polymorphic region corresponding to position 1661 of SEQ ID NO:6; and the polymorphic region corresponding to position 1662 of SEQ ID NO:6.
